(19) United States
(12) Patent Application Publication
(10) Pub. No.: US 2024/0320592 A1
(43) Pub. Date: Sep. 26, 2024

(54) PANDEMIC RESPONSE SUPPLY CHAIN SIMULATION SYSTEM AND METHOD

(71) Applicant: Logistics and Supply Chain MultiTech R&D Centre Limited, Hong Kong (HK)

(72) Inventors: Chin Chiu CHUNG, Hong Kong (HK); Chi Hung TONG, Hong Kong (HK); Kong SIT, Hong Kong (HK)

(21) Appl. No.: 18/187,154

(22) Filed: Mar. 21, 2023

(51) Int. Cl. G06Q 10/0637; G06Q 10/0639; G06Q 10/067; G06Q 10/08

(52) U.S. Cl. CPC G06Q 10/06375; G06Q 10/06395; G06Q 10/067; G06Q 10/08

(57) ABSTRACT

A pandemic response supply chain simulation system for simulating multiple scenarios during a pandemic comprising a computing apparatus comprising a memory unit and a processing unit arranged in communication with the memory unit, a user interface operatively coupled to the processing unit, the user interface configured to receive inputs from a user and present information to the user, the memory unit storing processing unit executable instructions that, the processing unit, configured to execute the instructions causing the system to receive a pandemic scenario related to a pandemic, receive one or more inputs via the user interface, receive one or more parameters related to the pandemic scenario, retrieve or receive one or more data related to previous pandemic scenarios, generate and/or load a supply chain operation model based on the inputs, parameters and the data related to previous pandemic scenarios, automatically apply the supply chain operation model to process the inputs and parameters, wherein the model is configured to simulate the pandemic scenario based on the inputs, parameters and the data related to the previous pandemic scenarios, generate one or more results of the simulation by the supply chain operational model, present on the user interface simulation results from the model.

### TECHNICAL FIELD

The present invention relates to a pandemic response supply chain simulation system for simulating multiple scenarios during a pandemic. The present invention further relates to a pandemic response supply chain simulation method.

### BACKGROUND

Tools for analytics and simulation of supply chains are well known. Supply chains for various goods and services are now global and often are very large in scale. Supply chains are also inter-dependent globally. Supply chains for drugs and other medications are large, complex and global in nature.

Pandemics or epidemics can be scenarios that can stress supply chains. Pandemic and epidemic scenarios can have an acute impact on the drug supply chain i.e., the medication supply chain. Such large-scale disease scenarios can make it very difficult to manage the distribution of medications since it is very difficult to predict surges in demand, distribution rates and other factors that can affect the distribution of medications. The COVID-19 pandemic that occurred recently has highlighted the challenges in medication supply chains.

There is a need for a system and method that provides analytics and simulation to mock different pandemic and epidemic scenarios to allow authorities and domain experts in policy to determine the impact of their decisions and allow for improve medication supply chain management.

### SUMMARY OF THE INVENTION

The present invention relates to a pandemic response supply chain simulation system and method. In particular, the present invention relates to a pandemic response medication supply chain simulation system and method. The supply chain simulation system as described herein allows a user e.g., governments, disease centres, health authorities to simulate multiple pandemic scenarios and determine the impact on the medication supply chain.

The simulation system allows users to assess the impact of their proposed decisions before implementing a decision. This simulation system provides a valuable tool for users e.g., governments, disease centres, health authorities to interrogate proposed decisions. The tool helps users e.g., governments, disease centres, health authorities to make better decisions around provisioning of medications during pandemic scenarios.

The simulation system further provides a tool for users to make for inclusive and equitable decisions around provisioning of medications. The simulation system allows for better medication supply chain management.

In one aspect the present invention relates to a pandemic response supply chain simulation system for simulating multiple scenarios during a pandemic comprising:
a computing apparatus comprising a memory unit and a processing unit arranged in communication with the memory unit,
a user interface operatively coupled to the processing unit, the user interface configured to receive inputs from a user and present information to the user,
the memory unit storing processing unit executable instructions that, the processing unit, configured to execute the instructions causing the system to:
receive a pandemic scenario related to a pandemic,
receive one or more inputs via the user interface,
receive one or more parameters related to the pandemic scenario,
retrieve or receive one or more data related to previous pandemic scenarios,
generate and/or load a supply chain operation model based on the inputs, parameters and the data related to previous pandemic scenarios,
automatically apply the supply chain operation model to process the inputs and parameters, wherein the model is configured to simulate the pandemic scenario based on the inputs, parameters and the data related to the previous pandemic scenarios,
generate one or more results of the simulation by the supply chain operational model,
present on the user interface simulation results from the model.

In an embodiment, the supply chain operational model is configured to simulate one or more effects on one or more elements of the supply chain based on the inputs, parameters for the pandemic scenario.

In an embodiment, the supply chain operation model may be a machine learning model that is trained to simulate one or more effects on one or more elements of the supply chain based on the inputs, parameters for the pandemic scenario.

Optionally the supply chain operation model is a neural network.

In an embodiment, the simulation results comprise numerical data defining the effects on one or more elements of the supply chain for the specified pandemic. The results may be presented as graphs, tables, charts, lists etc.

Optionally the system may automatically interpret the data and alarm accordingly or send messages to other systems based on the results interpretation. Alternatively, the results may be presented for interpretation by an expert e.g., a domain expert.

In an embodiment, the one or more effects relate to the effect on the supply of one or more medications during a predefined pandemic and wherein the inputs relate to facts about the one or more medications and the parameters relate to facts about the pandemic.

In one example the system is configured to:
receive changes to inputs and/or parameters related to the pandemic scenario via the user interface,
automatically update the supply chain operational model based on the received changes to the inputs and parameters,
apply the updated model to generate new results related to an updated simulation of the pandemic scenario.

In an embodiment, the system may be configured to update the supply chain operational model based on changes or updates to data related to the previous pandemic scenarios.

Optionally, the system is configured to:
apply a mathematical function to transform the data related to previous pandemic scenarios, and
normalise the transformed data for use in the supply chain operational model.

In an embodiment, data related to previous experiences may be posed as a question for processing an NLP model implemented by the system. Alternatively, previous results may be converted into numeric format or a format that is consistent for use in the operational model.

In an embodiment, the system may be configured to update the supply chain operational model in real time.

In an embodiment, the simulation results are indicative of a supply chain response during the defined pandemic.

In an embodiment, the system may be configured to generate the new results in real time based on changes to the inputs and/or parameters.

In one example the pandemic scenario is defined as a question, the processor being configured to interpret the question based on applying a natural language processing (NLP) model.

In one example the system is configured to:
generate inputs and/or parameters based on NLP processing of the question and,
utilise the generated inputs and/or parameters in the simulation.

In one example the system is configured to:
receive new inputs and parameters and/or a new scenario based on the results of the simulation and,
repeat the functions of the system described above for the new inputs and parameters and/or new simulation scenario.

In another aspect the present invention relates to a pandemic response supply chain simulation method comprising the steps of:
receiving a pandemic scenario related to a pandemic,
receiving one or more inputs via the user interface,
receiving one or more parameters related to the pandemic scenario,
retrieving or receiving one or more data related to previous pandemic scenarios,
generating and/or loading a supply chain operation model based on the inputs, parameters and the data related to previous pandemic scenarios,
automatically applying the supply chain operation model to process the inputs and parameters, wherein the model is configured to simulate the pandemic scenario based on the inputs, parameters and the data related to the previous pandemic scenarios,
generating one or more results of the simulation by the supply chain operational model,
presenting on the user interface simulation results from the model

In one example the method comprises the additional steps of:
receiving changes to inputs and/or parameters related to the pandemic scenario via the user interface,
automatically updating the supply chain operational model based on the received changes to the inputs and parameters, and;
applying the updated model to generate new results related to an updated simulation of the pandemic scenario.

In one example the method comprises:
applying a mathematical function to transform the data related to previous pandemic scenarios, and
normalising the transformed data for use in the supply chain operational model,
   updating the supply chain operational model based on changes or updates to data related to the previous pandemic scenarios.

Optionally the pandemic scenario is defined as a question, and the method comprises:
interpreting the question by applying a natural language processing (NLP) model,

generating inputs and/or parameters based on NLP processing of the question and,
utilising the generated inputs and/or parameters in the simulation.

In one example the method comprises the steps of:
receiving new inputs and parameters and/or a new scenario based on the results of the simulation and,
repeating the steps of the method described above for the new inputs and parameters and/or new simulation scenario.

The term “pandemic” as used herein encompasses a pandemic or epidemic or any other disease that infects a large number of people.

### BRIEF DESCRIPTION OF THE DRAWINGS

Embodiments of the pandemic response supply chain simulation system and method will now be described, by way of example, with reference to the accompanying drawings in which:

FIG. 1 illustrates a schematic diagram of a computing apparatus is arranged to be implemented as an example embodiment of a pandemic response supply chain simulation system.

FIG. 2 illustrates diagram of a pandemic response supply chain simulation method.

FIG. 3 illustrates an example method of changing or updating the scenario to and simulate the effect on the supply chain.

FIG. 4 illustrates an example of simulation results generated by the system and presented on the user interface.

FIG. 5 illustrates an updated scenario with updated inputs.

### DETAILED DESCRIPTION OF THE PREFERRED EMBODIMENTS

Referring to the figures example forms of a pandemic response supply chain simulation system and method for simulating multiple scenarios during a pandemic will be described. The present invention relates to a pandemic response supply chain simulation system for simulating supply of goods e.g., supply of medications during the multiple pandemic related scenarios. The pandemic scenarios may be changed or defined by a user. The system allows the user to simulate the supply chain for goods e.g., simulate if there will be enough medication for a specified population during a specific pandemic. Parameters related to the pandemic scenario are defined by the user and the system is configured to automatically provide results of the simulation regarding the supply of goods. The simulation is based on the inputs and parameters related to the defined scenario. The system may provide the result i.e., an outcome in real time allowing for improved decision making. The present invention further relates to a pandemic response supply chain simulation method that provides similar utility.

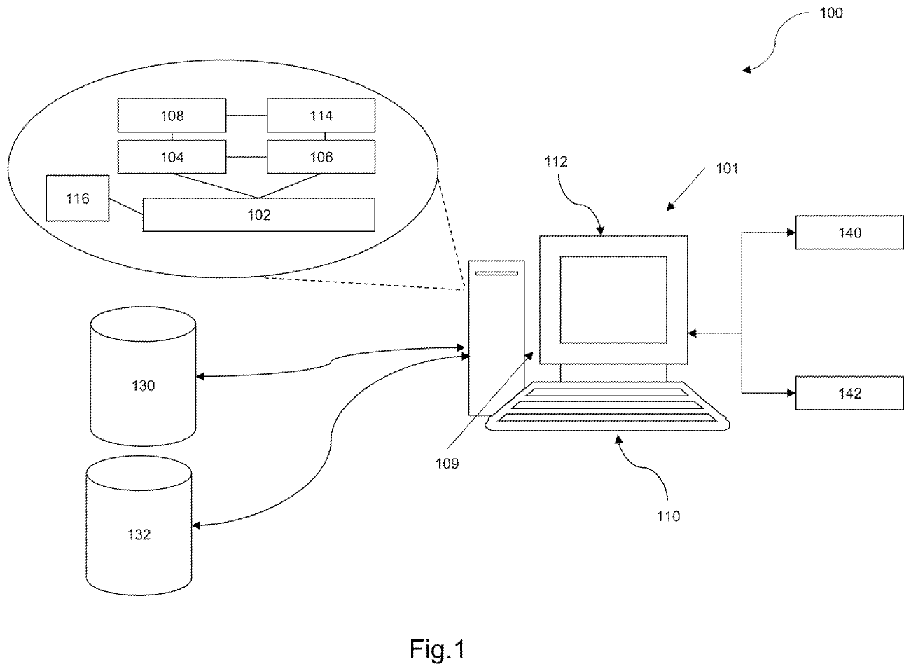

Referring to FIG. 1, an embodiment of the present invention is illustrated. This embodiment is arranged to provide a pandemic response supply chain simulation system 100 comprising: a computing apparatus comprising a memory unit and a processing unit arranged in communication with the memory unit, a user interface operatively coupled to the processing unit, the user interface configured to receive inputs from a user and present information to the user, the memory unit storing processing unit executable instructions that, the processing unit, configured to execute the instructions causing the system to: receive a pandemic scenario related to a pandemic, receive one or more inputs via the user interface, receive one or more parameters related to the pandemic scenario, retrieve or receive one or more data related to previous pandemic scenario s, generate and/or load a supply chain operation model based on the inputs, parameters and the data related to previous pandemic scenarios, automatically apply the supply chain operation model to process the inputs and parameters, wherein the model is configured to simulate the pandemic scenario based on the inputs, parameters and the data related to the previous pandemic scenarios, generate one or more results of the simulation by the supply chain operational model, present on the user interface simulation results from the model.

The supply chain operational model is configured to simulate one or more effects on one or more elements of the supply chain based on the inputs, parameters for the pandemic scenario. The supply chain operation model is a machine learning model that is trained to simulate one or more effects on one or more elements of the supply chain based on the inputs, parameters for the pandemic scenario. The simulation results comprise numerical data defining the effects on one or more elements of the supply chain for the specified pandemic. The system 100 is further configured to: receive changes to inputs and/or parameters related to the pandemic scenario via the user interface, automatically update the supply chain operational model based on the received changes to the inputs and parameters and apply the updated model to generate new results related to an updated simulation of the pandemic scenario.

In this example embodiment, the user interface and processing unit i.e. the system 100 are implemented by a computing apparatus 101 i.e. computing device, computer, computing system. The computing apparatus 101 may be implemented by any computing architecture, including portable computers, tablet computers, stand-alone Personal Computers (PCs), smart devices, Internet of Things (IOT) devices, edge computing devices, client/server architecture, “dumb” terminal/mainframe architecture, cloud-computing based architecture, or any other appropriate architecture, a smartphone or tablet. The computing apparatus 101 may be appropriately programmed to implement the invention.

In this embodiment, the system and method are arranged to allow a user to mock i.e., simulate different pandemic scenarios. The simulation system 100 provides a simulation related to supply of goods e.g., supply of medications. The system 100 allows a user to visualise the results of the simulation. The results of the simulation relate to the supply of goods in various pandemic scenarios. This allows a user to perform automatic and real time risk assessment. The simulation system allows users e.g., authorities or domain experts in health policy to study and consider the impact of their decisions in relation to supply of goods such as medications during a pandemic. The system also helps in analysis of risk assessment and logistics planning.

As shown in FIG. 1 there is a shown a schematic diagram of a computing apparatus 101 is arranged to be implemented as an example embodiment of a pandemic response supply chain simulation system 100. The system 100 may also comprise additional computing systems 140, 142. These computing systems are arranged in communication with the computing apparatus 101. The computing apparatus 101 i.e. the system 100 can access data from the other computing systems 140, 142 such as patient health data, patient vaccination rates, infection rates, transportation times from delivery systems etc.

In this embodiment the computing apparatus includes suitable components necessary to receive, store and execute appropriate computer instructions. The components may include a processing unit 102, including Central Processing Unit (CPU), Math Co-Processing Unit (Math Processor), Graphic Processing Unit (GPUs) or Tensor processing unit (TPUs) for tensor or multi-dimensional array calculations or manipulation operations, read-only memory (ROM) 104, random access memory (RAM) 106, and input/output devices such as disk drives 108.

The computing apparatus comprises a user interface 109 that may comprise input devices 110 and a display 112. The user interface 109 is operatively coupled to the processing unit 102. The user interface 109 is configured to receive inputs from a user and present information to the user. The input devices 110 may be any suitable input device to allow information to be inputted such as an Ethernet port, a USB port, etc or a keyboard or keypad. The display 112 allows information to be presented to a user such as a liquid crystal display, a light emitting display or any other suitable display and communications links 114.

The server 100 may include instructions that may be included in ROM 104, RAM 106 or disk drives 108 and may be executed by the processing unit 102. The memory unit e.g. ROM 104, or RAM 106 or disk drives 108 are programmed with executable instructions. The memory unit 104, 106, 108 is configured to store processing unit executable instructions. The processing unit is configured to execute the instructions causing the system to perform the steps of the method described later.

There may be provided a plurality of communication links 114 which may variously connect to one or more computing devices such as a server, personal computers, terminals, wireless or handheld computing devices, Internet of Things (IoT) devices, smart devices, edge computing devices. The communications link 114 may comprise a cellular modem e.g., a 3G, 4G or 5G modem. The communications link 114 may optionally also comprise Bluetooth and/or WiFi modules. Further the communications link 114 may comprise NFC unit with coils and appropriate electronics. At least one of a plurality of communications link may be connected to an external computing network through a telephone line or other type of communications link.

The computing apparatus 101 may include storage devices such as a disk drive 108 which may encompass solid state drives, hard disk drives, optical drives, magnetic tape drives or remote or cloud-based storage devices. The server 100 may use a single disk drive or multiple disk drives, or a remote storage service. The server 100 may also have a suitable operating system which resides on the disk drive or in the ROM of the computing apparatus 101.

The computer or computing apparatus 101 may also provide the necessary computational capabilities to operate or to interface with a machine learning network, such as a neural network, to provide various functions and outputs. The neural network may be implemented locally, or it may also be accessible or partially accessible via a server or cloud-based service. The machine learning network may also be untrained, partially trained or fully trained, and/or may also be retrained, adapted or updated over time.

The computing apparatus 101 comprises a model generator 116. The model generator 116 may be a hardware unit that is operatively coupled to the processing unit 102. Alternatively, the model generator 116 may be a software module that is incorporated into the processing unit 102.

The model generator 116 is configured to generate a supply chain operational model that is used to perform the simulation related to the pandemic scenario defined by a user. The model generator 116 may generate a model based on the inputs, parameters and the data related to previous pandemic scenarios. The model generator 116 may store multiple libraries and generate a custom operational model. Alternatively, the model generator may load one or multiple predefined models. The operational model used to perform the simulation is a machine learning model. In one example the model is a neural network.

The computing apparatus 101 further comprises multiple databases. The computing apparatus 101 comprises a previous pandemic scenario database 130. Database 130 stores data related to previous pandemic scenarios. The data related to previous pandemic scenarios can include the output from previous simulations. The data can include knowledge from past pandemic operations. The data can include inputs and parameters related to previous pandemic simulations in addition to the results of previous simulations.

The computing apparatus 101 further comprises a pandemic information database 132. The pandemic information database 132 can include various information related to a pandemic e.g., infection rates, population statistics etc. Further there could be information related to supply chain within the database 132.

In this embodiment, the pandemic response supply chain simulation system 100 for simulating multiple scenarios is used to perform a pandemic response supply chain simulation method 200. The system 100 and its components are used to perform the steps of the method.

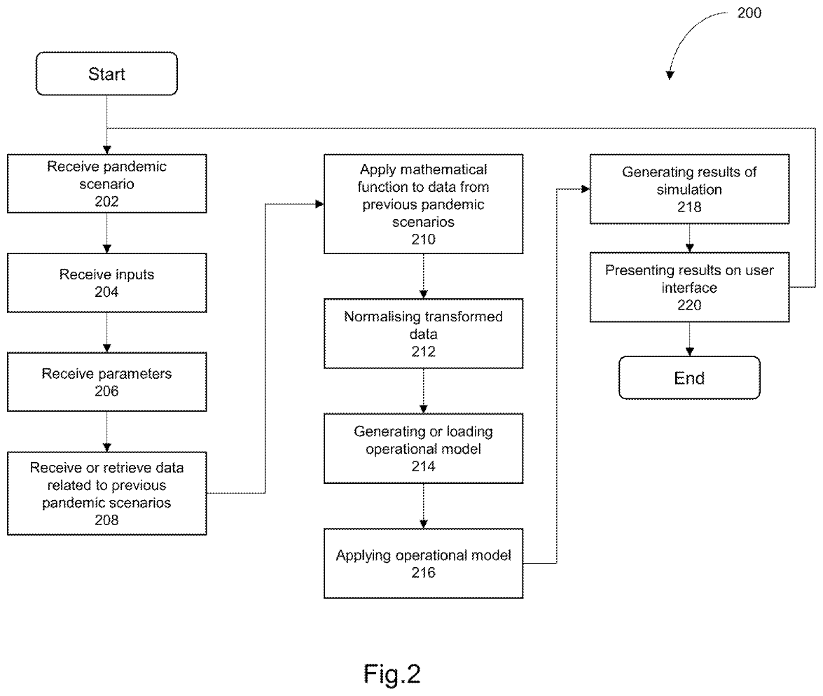

FIG. 2 illustrates a block diagram of the steps of method 200. The pandemic response supply chain simulation method 200 is used to simulate the effects on a supply chain based on multiple scenarios. In one example implementation the method 200 and system 100 are useful in determining if anti-pandemic supplies e.g., medications can be provided for a particular pandemic. Demand and supply anti-pandemic resources e.g., medications can fluctuate greatly. The system 100 and method 200 allow a user to simulate the impacts on medication supply for predefined pandemic scenarios.

The method 200 commences at step 202. Step 202 comprises receiving a pandemic scenario related to a pandemic. The scenario comprises a problem or goal statement. This goal statement is a scenario the user is interested in. The goal may be defined by the user and inputted via the user interface 109. For example, the goal statement may be a question such as for example: Can the existing stock level of the pandemic medications sustain through the programme plan in response to the growing demand for the current pandemic? What is booking levels fluctuate? How does it impact the supply and logistics? How many pandemic medication doses should be purchased for the next 3 months. The goal may define a pandemic scenario.

The pandemic scenario may be defined as a question. The processor 102 is configured to interpret the question based on applying a natural language processing (NLP) model. The NLP model may be stored in a memory unit and executed by the processor. The user may input the pandemic scenario as one or more questions. The NLP model is trained to process these questions.

Step 204 comprises the processor receiving one or more inputs via the user interface. Step 206 comprises the processor 102 receiving one or more parameters via the user interface. The parameters are related to the pandemic scenario. The inputs are related to the supply chain and more specifically to the goods.

Prior to steps 204, 206, the inputs and parameters are generated. The scenario question may be too general. The NLP model may further convert or determine one or more specific data analysis related questions. Alternatively, these questions may be generated by the user and inputted via the user interface. The NLP model is configured to generate the inputs and parameters-based processing the question i.e., the pandemic scenario. Optionally, in addition to processing the pandemic scenario the NLP model may further process more specific sub questions and identify the inputs and parameters. Inputs may relate to information about the goods, e.g., related to the medications. The parameters may relate to information about pandemic. These can be extracted from the questions provided via the user input automatically by processing the inputs with the NLP model.

Some example questions that can be used to identify or generate inputs and parameters can be: Can the existing stock level of BioNTech vaccines sustain for 3 months if the growth rate of the demand at CVCs is 20% per month? Or How many BioNTech vaccines be needed if the corresponding booking level keeps growing by an average of 10% per month? These questions are generally formulated by a human, but may be formulated by the processor executing the NLP model.

Step 208 comprises retrieving or receiving one or more data related to previous pandemic scenarios. The data about previous pandemic scenarios may relate to inputs, parameters associated with the previous pandemic scenarios. The data may also comprise results of the previous simulations associated with the previous pandemic scenarios. The data may be retrieved from the database 130.

Optionally the system may be configured to generate inputs and/or parameters based on NLP processing of the question and, utilise the generated inputs and/or parameters in the simulation.

Step 210 comprises applying a mathematical function to transform the data related to previous pandemic scenarios into a useable format. Step 212 comprises the step of normalising the transformed data for use in the supply chain operational model. The steps of transforming and normalising capitalise the data from the previous pandemic scenarios so that it can be used in simulating the new scenarios. This is advantageous as the model uses previous experiences to provide a more accurate simulation.

Step 214 comprises generating and/or loading a supply chain operational model based on the inputs, parameters and data related to previous pandemic scenarios. The operational model may be loaded or generated by the model generator 116. The model generator 116 is configured to generate the model by using appropriate libraries. Alternatively, the model generator 116 comprises a plurality of predefined models and the generator 116 selects the appropriate model.

Step 216 comprises automatically applying the supply chain operation model to process the inputs and parameters, wherein the model is configured to simulate the pandemic scenario based on the inputs, parameters and the data related to the previous pandemic scenarios.

Step 218 comprises generating one or more results of the simulation by the supply chain operational model. Step 220 comprises presenting on the user interface simulation results from the model. The simulation results comprise numerical data defining the effects on one or more elements of the supply chain for the specified pandemic. Method 200 may be repeated.

The supply chain operational model is configured to simulate one or more effects on one or more elements of the supply chain based on the inputs, parameters for the pandemic scenario. The one or more effects relate to the effect on the supply of one or more medications during a predefined pandemic and wherein the inputs relate to facts about the one or more medications and the parameters relate to facts about the pandemic. The effects relate to a response to the initial questions or queries posed. The simulation results are indicative of a supply chain response during the defined pandemic.

Optionally, the simulation results comprise numerical data defining the effects on one or more elements of the supply chain for the specified pandemic.

In one example, the supply chain operation model is a machine learning model that is trained to simulate one or more effects on one or more elements of the supply chain based on the inputs, parameters for the pandemic scenario. Preferably the supply chain operation model is a neural network. Alternatively, other suitable machine learning models can be used.

The operational model used is preferably customised based on the various inputs and parameters originating from the scenario questions, as explained earlier. The model is customised to provide a simulation and can be changed or updated by changing the scenario question, changing the inputs and changing the parameters. Any one or more of these elements can be changed to change the operational model. The system is configured to update the supply chain operational model based on changes or updates to data related to the previous pandemic scenarios. Optionally, the system is configured to update the supply chain operational model in real time.

The method 200 can be repeated for new inputs and new parameters. For example, the system 100 is configured to receive new inputs and parameters and/or a new scenario based on the results of the simulation. The system 100 may be configured to repeat the steps of method 200 for the new inputs and parameters and/or new simulation scenario. Method 200 can be repeated to simulate new scenarios. Further the results may yield new inputs or new parameters that can be tested.

All results, inputs and parameters used may be stored in one or more of the databases 130, 132. For example, the results and current scenario data may be stored in database 130 for accessing in the future.

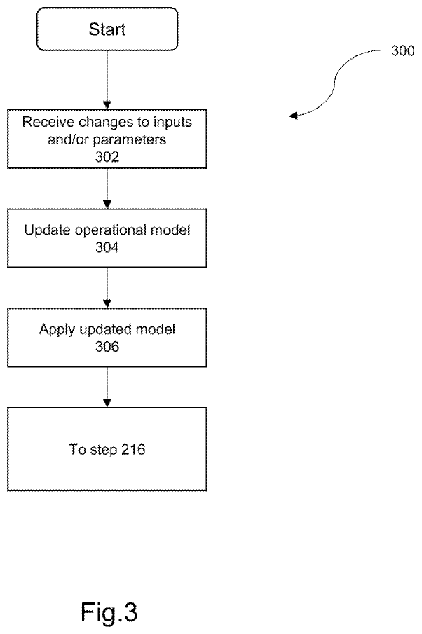

The system 100 is further advantageous because it allows a user to change the scenario and change inputs and/or parameters and generate a new simulation i.e., generate new results. This allows a user to test multiple scenarios and the effect on the supply chain rapidly and automatically on the supply chain. The question or questions related to the scenario can be changed via the user interface 109. FIG. 3 illustrates an example method of changing or updating the scenario 300 to and simulate the effect on the supply chain.

Step 302 comprises receiving changes to inputs and/or parameters related to the pandemic scenario via the user interface. Step 304 comprises automatically update the supply chain operational model based on the received changes to the inputs and parameters. The model may be updated by the processing unit 102. The model may be changed to improve its accuracy. Step 306 comprises applying the updated model to generate new results related to an updated simulation of the pandemic scenario. The method may then proceed to step 216 and continue with steps 216 to 220.

The system 100 may further be configured to generate the new results in real time based on changes to the inputs and/or parameters.

In an example the question posed by the user is “can the existing stock level of BioNTech vaccines sustain for three months if the growth rate of the demand is 1% per day”. Such a question may be processed by the NLP model to identify the inputs as i) stock level in 3 months, ii) growth rate of 1% per day, iii) the initial number of BioNTech vaccines available, iv) vaccine type, v) vaccine operation mode (e.g., CVC only, CVC and private clinic), vi) vaccine ordering rates. The parameters could be identified as i) vaccine rate, ii) number of infected, iii) total population, iv) disease affecting people, v) infection rate. Additional inputs or parameters may be inputted by the user via the user interface 109. The inputs generally relate to information about the vaccine while parameters relate to information about the pandemic and its effect on the population.

In addition to the above results of older simulations can be used too. For example, the previous vaccination rate, stock level, population of vaccinated vs unvaccinated may be data of previous simulations. Further data can also relate to the outputs of the previous simulations e.g., the vaccine stock of X number did not last a population of Y number when the growth rate was 3%.

The operational model can be applied to the above inputs and parameters to simulate if the existing stock level of BioNTech vaccines can be sustained for 3 months. This is in relation to the pandemic scenario of “whether the current vaccine stock is enough to sustain for the next 3 months?”. The inputs and parameters define the scenario. The inputs and parameters are further data that is used mathematically define the scenario such that the system can process it and automatically generate results. The results are numerical results and presented on the user interface 109 for the user to interpret. The results may further comprise tables, graphs, lists, charts etc. that are automatically generated by the processing unit 102 and presented to the user.

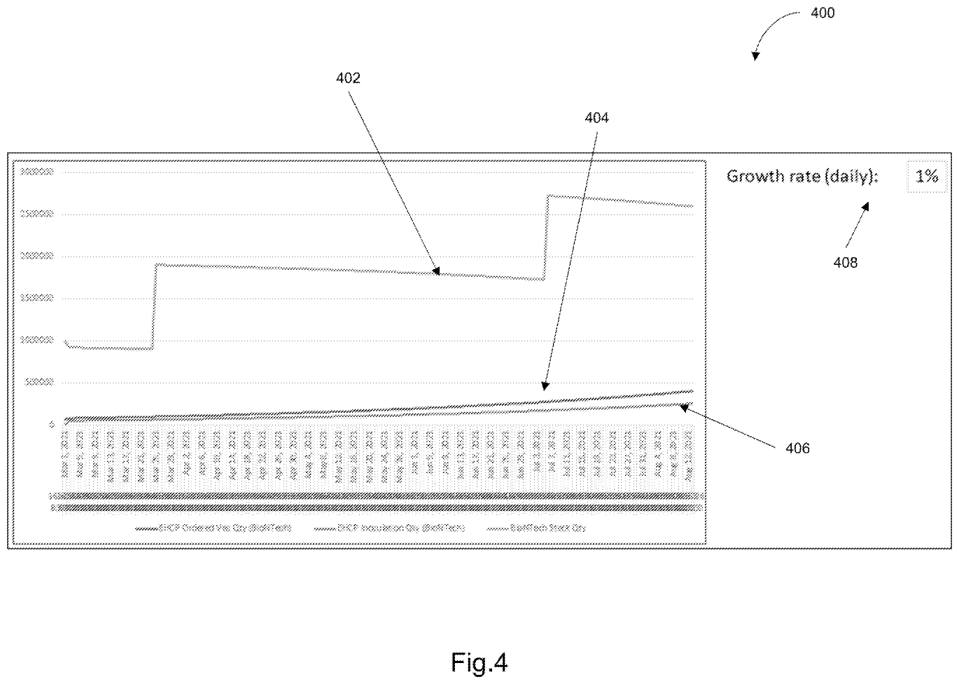

FIG. 4 illustrates an example of simulation results generated by the system 100 and presented on the user interface. The results screen 400 illustrates the answer to the question posed “can the existing stock level of BioNTech vaccines sustain for three months if the growth rate of the demand is 1% per day”. Referring to FIG. 4, the simulation result indicates that the current stock of vaccines will be sufficient. The results are presented on a graph. The simulation is operated over 3 months and the results are shown in 4 day intervals along the X axis. The Y axis relates to the quantity of vaccines.

The growth rate 408 is illustrated on screen 400. The BioNTech vaccine stocks indicated by line 402. As can be seen the stock levels increase as more vaccines are either ordered or manufactured. The ordering quantities are shown by line 404. The inoculation rate is shown by line 406. As can be seen in FIG. 4, the stock of vaccines 402 is always greater than the ordered stock and inoculation rate, even if the inoculation rate is growing by 1% per day. Line 406 indicates a inoculation growth at 1% growth rate. The question is clearly answered that the stock of vaccines is sufficient. As can be seen from the results screen 400 the current stock can last over 3 months.

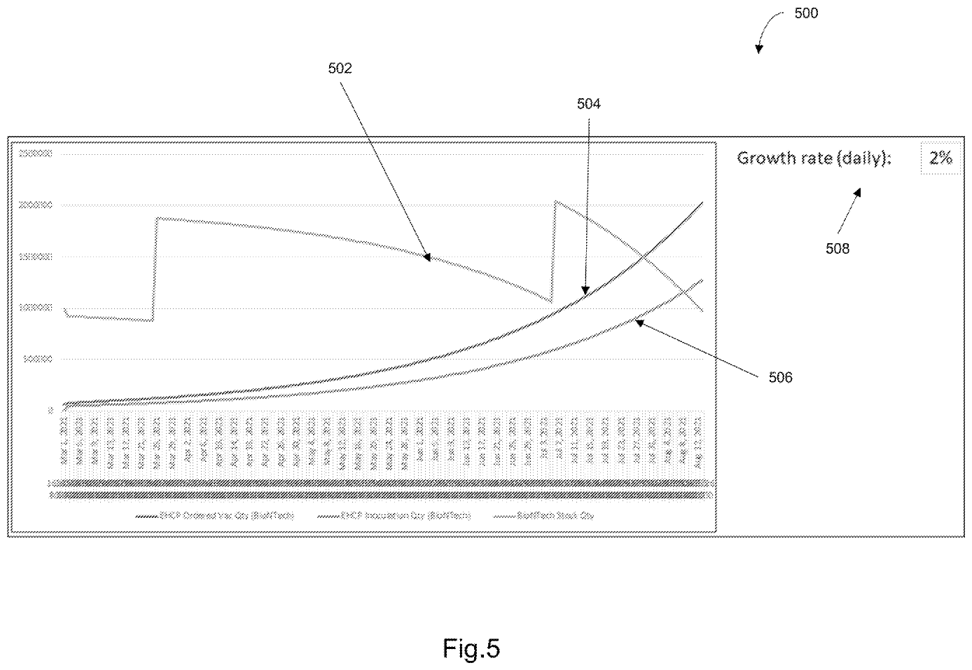

FIG. 5 illustrates an updated scenario with updated inputs. The input changed is the daily growth rate which is now 2% instead of 1%. A user can change inputs live and optionally in real time. The simulation is updated based on the changes to the inputs. Other inputs and parameters may be changed. Additionally, or alternatively, the entire scenario may be changed. FIG. 5 illustrates an updated results screen 500. As seen can from screen 500, the growth rate graph 506 illustrates growth rate of 2% of inoculation growth rate. The growth rate 2% is shown on the screen 500 by feature 508. The stock levels shown by line 502 are affected more and drop as compared to line 402. The ordering quantities shown by line 504 increase in line with the inoculation rate at 2% too. As can be seen from the results screen 500, the inoculation numbers and ordering numbers exceed the stock. Based on the simulation results of FIG. 5, the BioNTech vaccine stocks will last until 29 July.

The results may be represented visually and interpreted by domain experts who understand the outputs. Domain experts can read and understand results visually presented. Experts can use the simulation system to simulate various scenarios and interpret the results and link the results to the goal i.e., the overall scenario. The simulation can result in new questions being formulated and creation of new knowledge. If the results are not satisfactory, the system 100 is adaptable and a user can update the scenario or inputs or parameters. Additional inputs and parameters may be generated, automatically, by the system 100 based on the results in order to address the initial scenario.

The system 100 is advantageous because it provides a simple to use tool that allows users to make better policy decisions. The system 100 allows experts to assess the impact of their decisions and simulate the impact on the wider population. The system 100 is particularly useful as a tool to assess the impact of pandemics on various supply chains e.g., vaccines, food, water, medicines etc. The system 100 is robust and allows users to simulate multiple different scenarios. The system 100 provides a real time, automated tool to simulate impacts of pandemics on supply chains. The system 100 also allows a user to change scenarios to assess the impacts of multiple different scenarios. The system 100 is adaptable and allows a user to change inputs and simulate the impact of various or different input conditions. This provides a robust tool that is easy to use and operates in real time allowing better decision making.

The system 100 is further advantageous since it can be implemented in a smartphone or tablet and therefore is a portable system that can be used anywhere e.g., at ground zero of a pandemic or at clinics or emergency medical posts etc. The system 100 is portable and operates in substantially real time allowing for multiple scenarios to be tested in a short amount of time leading to improved decision making.

The system 100 and method 200 as described herein is described in reference to pandemics and the impact of pandemics but the tool may be used for simulating the impacts on supply chains due to other events such as for example natural disasters. The present invention could be applied to simulate the effect of natural disasters or manmade disasters or war or famine or such major events.

Although not required, the embodiments described with reference to the Figures can be implemented as an application programming interface (API) or as a series of libraries for use by a developer or can be included within another software application, such as a terminal or personal computer operating system or a portable computing device operating system. Generally, as program modules include routines, programs, objects, components and data files assisting in the performance of particular functions, the skilled person will understand that the functionality of the software application may be distributed across a number of routines, objects or components to achieve the same functionality desired herein.

It will also be appreciated that where the methods and systems of the present invention are either wholly implemented by computing system or partly implemented by computing systems then any appropriate computing system architecture may be utilised. This will include stand-alone computers, network computers and dedicated hardware devices. Where the terms “computing system” and “computing device” are used, these terms are intended to cover any appropriate arrangement of computer hardware capable of implementing the function described.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Any reference to prior art contained herein is not to be taken as an admission that the information is common general knowledge, unless otherwise indicated.

The phrases ‘computer-readable medium’ or ‘machine-readable medium’ as used in this specification and claims should be taken to include, unless the context suggests otherwise, a single medium or multiple media. Examples of multiple media include a centralised or distributed database and/or associated caches. These multiple media store the one or more sets of computer executable instructions. The phrases ‘computer-readable medium’ or ‘machine-readable medium’ should also be taken to include any medium that is capable of storing, encoding or carrying a set of instructions for execution by a processor of a computing device and that cause the processor to perform any one or more of the methods described herein.

The computer-readable medium is also capable of storing, encoding or carrying data structures used by or associated with these sets of instructions. The phrases ‘computer-readable medium’ and ‘machine readable medium’ include, but are not limited to, portable to fixed storage devices, solid-state memories, optical media or optical storage devices, magnetic media, and/or various other mediums capable of storing, containing or carrying instruction (s) and/or data. The ‘computer-readable medium’ or ‘machine-readable medium’ may be non-transitory.

Also, it is noted that the embodiments may be described as a process that is depicted as a flowchart, a flow diagram, a structure diagram, or a block diagram. Although a flowchart may describe the operations as a sequential process, many of the operations can be performed in parallel or concurrently. In addition, the order of the operations may be rearranged. A process is terminated when its operations are completed. A process may correspond to a method, a function, a procedure, a subroutine, a subprogram, etc., in a computer program. When a process corresponds to a function, its termination corresponds to a return of the function to the calling function or a main function.

Furthermore, embodiments may be implemented by hardware, software, firmware, middleware, microcode, or any combination thereof. When implemented in software, firmware, middleware or microcode, the program code or code segments to perform the necessary tasks may be stored in a machine-readable medium such as a storage medium or other storage (s). A processor may perform the necessary tasks. A code segment may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

One or more of the components and functions illustrated the figures may be rearranged and/or combined into a single component or embodied in several components without departing from the scope of the invention. Additional elements or components may also be added without departing from the scope of the invention. Additionally, the features described herein may be implemented in software, hardware, as a business method, and/or combination thereof.

In its various aspects, embodiments of the invention can be embodied in a computer-implemented process, a machine (such as an electronic device, or a general-purpose computer or other device that provides a platform on which computer programs can be executed), processes performed by these machines, or an article of manufacture.

1. A pandemic response supply chain simulation system for simulating multiple scenarios during a pandemic comprising:
a computing apparatus comprising a memory unit and a processing unit arranged in communication with the memory unit,
a user interface operatively coupled to the processing unit, the user interface configured to receive inputs from a user and present information to the user,
the memory unit storing processing unit executable instructions that, the processing unit, configured to execute the instructions causing the system to:
receive a pandemic scenario related to a pandemic,
receive one or more inputs via the user interface,
receive one or more parameters related to the pandemic scenario,
retrieve or receive one or more data related to previous pandemic scenarios,
generate and/or load a supply chain operation model based on the inputs, parameters and the data related to previous pandemic scenarios,
automatically apply the supply chain operation model to process the inputs and parameters, wherein the model is configured to simulate the pandemic scenario based on the inputs, parameters and the data related to the previous pandemic scenarios,
generate one or more results of the simulation by the supply chain operational model,
present on the user interface simulation results from the model.

2. A pandemic response supply chain simulation system as per claim 1, wherein the supply chain operational model is configured to simulate one or more effects on one or more elements of the supply chain based on the inputs, parameters for the pandemic scenario.

3. A pandemic response supply chain simulation system as per claim 1, wherein the supply chain operation model is a machine learning model that is trained to simulate one or more effects on one or more elements of the supply chain based on the inputs, parameters for the pandemic scenario.

4. A pandemic response supply chain simulation system as per claim 2, wherein the supply chain operation model is a neural network.

5. A pandemic response supply chain simulation system as per claim 2, wherein the simulation results comprise numerical data defining the effects on one or more elements of the supply chain for the specified pandemic.

6. A pandemic response supply chain simulation system as per claim 5, wherein the one or more effects relate to the effect on the supply of one or more medications during a predefined pandemic and wherein the inputs relate to facts about the one or more medications and the parameters relate to facts about the pandemic.

7. A pandemic response supply chain simulation system as per claim 1, wherein the system is configured to:
receive changes to inputs and/or parameters related to the pandemic scenario via the user interface,
automatically update the supply chain operational model based on the received changes to the inputs and parameters,
apply the updated model to generate new results related to an updated simulation of the pandemic scenario.

8. A pandemic response supply chain simulation system as per claim 2, wherein the system is configured to update the supply chain operational model based on changes or updates to data related to the previous pandemic scenarios.

9. A pandemic response supply chain simulation system as per claim 1, wherein the system is configured to:
apply a mathematical function to transform the data related to previous pandemic scenarios, and
normalise the transformed data for use in the supply chain operational model.

10. A pandemic response supply chain simulation system as per claim 7, wherein the system is configured to update the supply chain operational model in real time.

11. A pandemic response supply chain simulation system as per claim 1, wherein simulation results are indicative of a supply chain response during the defined pandemic.

12. A pandemic response supply chain simulation system as per claim 7, wherein the system is configured to generate the new results in real time based on changes to the inputs and/or parameters.

13. A pandemic response supply chain simulation system as per claim 1, wherein the pandemic scenario is defined as a question, the processor being configured to interpret the question based on applying a natural language processing (NLP) model.

14. A pandemic response supply chain simulation system as per claim 13, wherein the system is configured to:
generate inputs and/or parameters based on NLP processing of the question and,
utilise the generated inputs and/or parameters in the simulation.

15. A pandemic response supply chain simulation system as per claim 1, wherein the system is configured to:
receive new inputs and parameters and/or a new scenario based on the results of the simulation and,
repeat the functions of claim 1 for the new inputs and parameters and/or new simulation scenario.

16. A pandemic response supply chain simulation method comprising the steps of:
receiving a pandemic scenario related to a pandemic,
receiving or more inputs via the user interface,
receiving one or more parameters related to the pandemic scenario,
retrieving or receiving one or more data related to previous pandemic scenarios,
generating and/or loading a supply chain operation model based on the inputs, parameters and the data related to previous pandemic scenarios,
automatically applying the supply chain operation model to process the inputs and parameters, wherein the model is configured to simulate the pandemic scenario based on the inputs, parameters and the data related to the previous pandemic scenarios,
generating one or more results of the simulation by the supply chain operational model,
presenting on the user interface simulation results from the model.

17. A pandemic response supply chain simulation method as per claim 16, comprising the additional steps of:
receiving changes to inputs and/or parameters related to the pandemic scenario via the user interface,
automatically updating the supply chain operational model based on the received changes to the inputs and parameters, and;
applying the updated model to generate new results related to an updated simulation of the pandemic scenario.

18. A pandemic response supply chain simulation method as per claim 16, wherein the method comprises:
applying a mathematical function to transform the data related to previous pandemic scenarios, and
normalising the transformed data for use in the supply chain operational model,
updating the supply chain operational model based on changes or updates to data related to the previous pandemic scenarios.

19. A pandemic response supply chain simulation method as per claim 16, wherein the pandemic scenario is defined as a question, the method comprises:
interpreting the question by applying a natural language processing (NLP) model,
generating inputs and/or parameters based on NLP processing of the question and,
utilising the generated inputs and/or parameters in the simulation.

20. A pandemic response supply chain simulation method as per claim 16, comprising the steps of:
receiving new inputs and parameters and/or a new scenario based on the results of the simulation and,
repeating the functions of claim 16 for the new inputs and parameters and/or new simulation scenario.
